(19) United States
(12) Patent Application Publication
(10) Pub. No.: US 2002/0098994 A1
(43) Pub. Date: Jul. 25, 2002

(54) One time use disposable paper soap and method of making

(76) Inventor: Alam Zafar, Queens, NY (US)

Correspondence Address: ALAM ZAFAR, 8100, Shore Front Parkway #12K, Rockaway Beach, Queens, NY, 11693, US

(21) Appl. No.: 09/769,000

(22) Filed: Jan. 22, 2001

(51) Int. Cl. C11D 17/00; A61K 7/50

(52) U.S. Cl. 510/140

(57) ABSTRACT

A one time use disposable paper soap. The soap includes a substrate, a cleaning composition, and a holder. The disposable paper is dissolvable in water. The cleansing composition is impregnated in the substrate so as to form a dry and cleanser impregnated substrate is not laid hold on to prevent exposure and evaporation of the cleansing composition, since the and cleanser impregnated substrate is dry. The holder removably holds at least one stacked dry and cleanser impregnated substrate. When the dry and cleanser impregnated substrate is removed from the holder and subjected to water, the substrate dissolves leaving the cleansing composition dissolved in the water for cleansing.

### BACKGROUND OF THE INVENTION

1. Field of the Invention

The present invention relates to a soap. More particularly, the present invention relates to a one time use disposable paper soap and method of making.

2. Description of the Prior Art

Nowadays public places are equipped with regular soap bars or liquid soap jars. Both of these situations are unhygienic.

Everyday a large number of people use the same bar or the same jar to wash their hands. Some are healthy, and some are not. Some of them might carry germs which are contagious. In some cases these germs may stay and transmit to other people later on. In some instances, people even spread the mess all over the places, especially when a number of children use the same bathroom.

Women with long nails might experience difficulty when using soap bars or jars. In addition, germs may go under their nails and stay there. Later on, these germs may be transmitted to themselves while eating or transmitted to their children while hugging and kissing.

Furthermore, in most of the cases, children do not take the preventive measures which are needed while using public bathrooms.

In a famous program named 20/20 that aired on Jul. 12, 1999 on ABC channel 7, it was claimed that the inadequacies and unhygienic conditions in public bathrooms spread 58% of the germs in the community.

Numerous innovations for cleansing substrates have been provided in the prior art that will be described. Even though these innovations may be suitable for the specific individual purposes to which they address, however, they differ from the present invention.

For example, U.S. Pat. No. 4,045,364 to Richter teaches a disposable paper with a slightly abrasive surface impregnated with an iodophor and detergent that is an excellent germicidal pre-wash in the surgical scrub routine or in any situation where it is important to impede the growth of microorganisms. By using substantially dry impregnated papers the product may be packaged and stored for at least 18 months without undue deterioration. In the presence of undue moisture, the paper degrades too fast for adequate storage.

### SUMMARY OF THE INVENTION

Accordingly, an object of the present invention is to provide a one time disposable paper soap and method of making that avoids the disadvantages of the prior art.

Another object of the present invention is to provide a one time use disposable paper soap and method of making that is simple and inexpensive to manufacture.

Still another object of the present invention is to provide a one time use disposable paper soap and method of making that is simple to use.

Briefly stated, yet another object of the present invention is to provide a one time use disposable paper soap. The soap includes a substrate, a cleansing composition, and a holder. The tissue paper which is coated with soap is dissolvable in water. The cleansing is impregnated in the substrate in the substrate so as to form a dry and cleanser impregnated substrate. The dry and cleanser impregnated substrate is not laid hold on to prevent exposure and evaporation of the cleansing composition, since the dry and cleanser impregnated substrate is dry. The holder removably holds at least one stacked dry and cleanser impregnated substrate. When the dry and cleanser impregnated substrate is removed from the holder and subjected to water, the substrate dissolves leaving the cleansing composition dissolved in the water for cleansing.

The novel features which are considered characteristic of the present invention are set forth in the appended claims. The invention itself, however, both as to its construction and its method of operation, together with additional objects and advantages thereof, will be best understood from the following description of the specific embodiments when read and understood in connection with the accompanying drawing.

### BRIEF DESCRIPTION OF THE DRAWING

The FIGURE of the drawing is briefly described as follows:

FIG. 1A is a diagrammatic perspective view of the present invention:

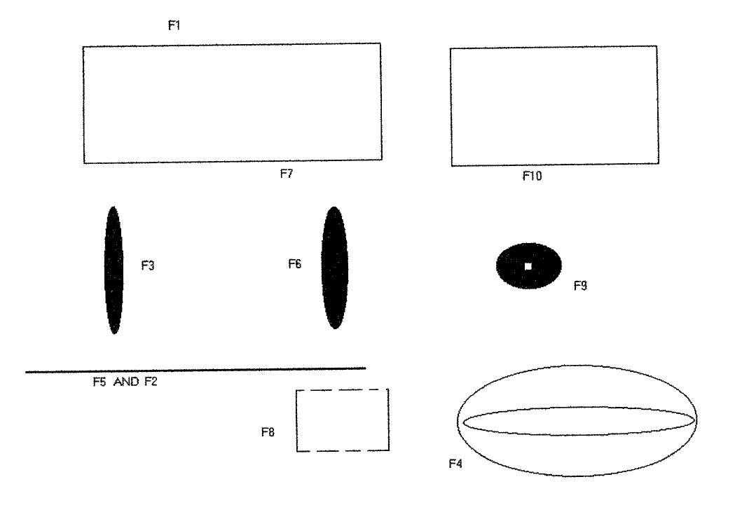

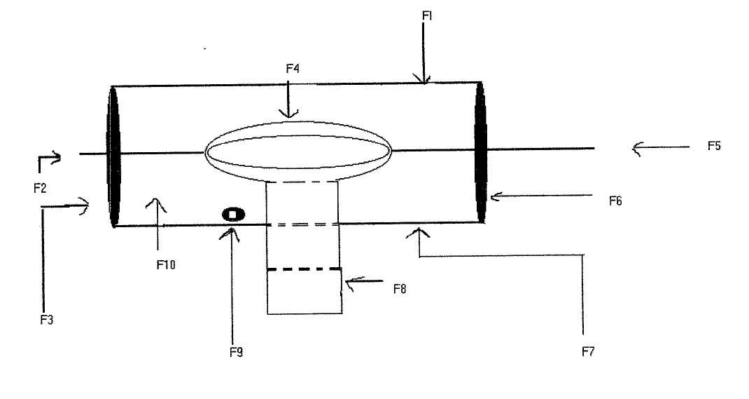

F1—COVER OF PLASTIC BOX

F2—PLASTIC ROD

F3—PLASTIC BOX HOLDER

F4—DISPOSABLE PAPER SOAP ROLL

F5—PLASTIC ROD

F6—PLASTIC BOX HOLDER

F7—COVER OF PLASTIC BOX

F8—ONE TIME USE DISPOSABLE PAPER SOAP

F9—LOCK TO OPEN THE PLASTIC BOX

F10—PLASTIC BOX

### LIST OF REFERENCE NUMERRALS UTILIZED IN THE DRAWING

1 one time use disposable paper soap of the present invention

2 substrate

3 cleansing composition

4 dry and cleanser impregnated substrate

5 holder

6 cover of holder 5

7 back portion of cover 6 of holder 5

8 impregnating station

9 wet and cleanser impregnated substrate

10 tissue paper

11 drying station

12 microwaves

13 cutting station

14 individual dry and cleanser impregnated substrates

15 stacking station

16 stack of individual dry and cleanser impregnated substrates

17 packaging station

18 tissue box

### STILL MORE DESCRIPTION

We invented disposable a paper soap for the use in public bathrooms such as in hospitals, schools, offices, restaurants, etc. It is in many different colors and fragrances. It cannot harm anyone in any way. It is very safe for personal hygiene. This soap is against a broad spectrum of diseases such as HIV/AIDS. It is further more particular object of the invention to provide a readily available premoistened, prepackaged one time use disposable paper soap disinfectant, including hazardous and often contageous contaminants, such as those known as the HIV-1 and AIDS viruses.

1. A one time use disposable paper soap, comprising:
A] A substrate being dissolvable in water;
B] A cleansing composition impregnated in said substrate so as to form a dry and cleanser impregnated subtrated; said dry and cleanser impregnated substrate being not laid hold on to prevent exposure and evaporation of said cleansing composition, since said dry and cleanser impregnated substrate is dry; and
C] A holder removably holding at least one stacked said dry and cleanser impregnated substrate, and when said dry and cleanser impregnated substrate is removed from said holder and subjected to water, said substrate dissolves leaving said cleansing composition dissolved in water for cleansing.
